(19) United States
(12) Patent Application Publication
(10) Pub. No.: US 2022/0227855 A1
(43) Pub. Date: Jul. 21, 2022

(54) COMPOSITIONS AND METHODS FOR TREATMENT OF ANGIOGENESIS RELATED DISEASES

(71) Applicant: The University of Massachusetts, Boston, MA (US)

(72) Inventors: Divyabharathy TSIROS, Dracut, MA (US); Matthew NUGENT, Bedford, MA (US)

(21) Appl. No.: 17/606,173

(22) PCT Filed: Apr. 22, 2020
(86) PCT No.: PCT/US20/29232

§ 371 Date: Oct. 25, 2021

Related U.S. Application Data
(60) Provisional application No. 62/838,491, filed on Apr. 25, 2019.

(51) Int. Cl. C07K 16/22; A61K 47/61; A61K 47/68

(52) U.S. Cl. CPC C07K 16/22; A61K 47/61; C07K 2317/76; A61K 47/6845; C07K 2317/24; A61K 47/6807

(57) ABSTRACT

Described herein is a method of treating a subject in need of treatment for pathological angiogenesis including administering to the subject a glycosaminoglycan linked either covalently or noncovalently to an anti-VEGF antibody, an anti-VEGF antibody fragment, an anti-VEGF protein, an anti-VEGF peptide, or an anti-VEGF aptamer. Also included is a glycosaminoglycan linked either covalently or noncovalently to an anti-VEGF antibody, an anti-VEGF antibody fragment, an anti-VEGF protein, an anti-VEGF peptide, or an anti-VEGF aptamer.

### CROSS-REFERENCE TO RELATED APPLICATIONS

This application claims priority to U.S. Provisional Application 62/838,491 filed on Apr. 25, 2019, which is incorporated herein by reference in its entirety.

### STATEMENT REGARDING FEDERALLY SPONSORED RESEARCH & DEVELOPMENT

This invention was made with government support under 1R15GM120702-01 awarded by the National Institutes of Health. The government has certain rights in the invention.

### FIELD OF THE DISCLOSURE

The present disclosure is related to compositions and methods for the treatment of pathological angiogenesis.

### BACKGROUND

Angiogenesis is a highly controlled process that involves a complex interplay between angiogenic and anti-angiogenic signals. Vascular endothelial growth factor-A (VEGF) is a major mediator of angiogenesis through its ability to bind to and activate receptors, specifically VEGF receptor 2 (VEGFR-2), on the surface of endothelial cells to signal cell migration, proliferation, differentiation and survival. VEGF-VEGFR-2 interactions are further enhanced by the presence of heparin/heparan sulfate (HS) proteoglycans (HSPGs). Heparin is produced in mast cells, while HS is found on cell surfaces and in the extracellular matrix (ECM) linked to proteins as HSPGs. Heparin/HS are known to be involved in many biological processes through the ability to interact with numerous proteins such as growth factors, cytokines, cell surface proteins, enzymes and ECM proteins. Within the VEGF system, heparin/HS has been shown to participate in modulating binding interactions at many levels. Heparin/HS directly binds to VEGF, enhances the affinity of VEGF binding to VEGFR-2, and binds to the VEGF binding proteins: VEGF receptor 1, neuropilin 1, and fibronectin (FN). Thus, heparin/HS has the potential to modulate VEGF activity through a variety of mechanisms.

In 1997, a humanized version of an anti-VEGF monoclonal antibody, bevacizumab, commercially known as Avastin®, was developed to bind VEGF and block its binding to VEGFR-2. Avastin® was shown to be effective at reducing angiogenesis in many clinical settings leading to FDA approval for the treatment of cancers such as ovarian cancer, advanced cervical cancer, metastatic renal cell carcinoma, recurrent glioblastoma, advanced nonsquamous non-small cell lung cancer, and metastatic colorectal cancer. However, Avastin® does not prevent VEGF binding to heparin or neuropilin 1, another cell-surface VEGF binding protein. Neuropilin has been shown to enhance VEGF binding to its signaling receptor, VEGFR-2. Avastin® was found to form multimeric complexes with VEGF as well as bind Fey receptors in the presence of heparin. This in turn activates platelet aggregation as well as a cell death cascade. The ability of VEGF to bind heparin and neuropilin in the presence of Avastin® is believed to limit VEGF clearance by Avastin®. HSPGs and neuropilin on endothelial cell surfaces and within the ECM are believed to trap VEGF, thus, limiting the effectiveness of Avastin®.

In addition to HSPGs, VEGF also binds to the ECM protein FN within the hep 2 domain (Type III domains 12-14) and this interaction has been demonstrated to enhance the angiogenic activity of VEGF. Growth factor interactions with ECM are associated with localized targeted signaling. In addition, FN interaction with VEGF is able to facilitate a synergistic binding complex with VEGFR-2 and αvβ3 integrin which is known to potentiate the angiogenic activity. Heparin/HS also hinds transiently to the hep 2 domain of FN causing a rearrangement of FN leading to exposure of the VEGF binding site. The ability of heparin/HS to catalyze the conversion of FN from a closed to an open conformation allows VEGF and other growth factors to decorate the ECM and locally guide cell response. Interestingly, the binding of VEGF to FN is enhanced at acidic pH suggesting that this process would be more pronounced in hypoxic and locally acidic extracellular tissue environments. While the ability of Avastin® to modulate a variety of VEGF binding events has been explored, little is known about how VEGF-FN interactions and changes in extracellular pH might influence the activity of Avastin®.

What is needed are novel methods of enhancing the ability of Avastin® to influence. VEGF binding to FN and to the ECM of endothelial cells.

### BRIEF SUMMARY

In one aspect, a method of treating a subject in need of treatment for pathological angiogenesis comprises administering to the subject a glycosaminoglycan linked either covalently or noncovalently to an anti-VEGF antibody, an anti-VEGF antibody fragment, an anti-VEGF protein, an anti-VEGF peptide, or an anti-VEGF aptamer.

In another aspect, a composition comprises a glycosaminoglycan linked either covalently or noncovalently to an anti-VEGF antibody, an anti-VEGF antibody fragment, an anti-VEGF protein, an anti-VEGF peptide, or an anti-VEGF aptamer.

### BRIEF DESCRIPTION OF THE DRAWINGS

FIGS. 1A and B show that Avastin® partially inhibits VEGF binding to the ECM of PAEC EV and FLK cells. FIG. 1A shows ¹²⁵I-VEGF (10 ng/mL) incubated with PAEC EV and ELK cells in the presence and absence of Avastin® nM) at pH 6.5 for 2 h at 4° C., and the ¹²⁵I-VEGF bound to ECM and cell surface were extracted and measured as described under Methods. The level of VEGF hound per well in each fraction are presented as the average of triplicate determinations±SD. FIG. 1B shows that to study the effects of components found in the ECM, the PAEC EV and ELK cells were pretreated with either heparin (10 μg/mL) to expose VEGF binding sites on FN or buffer for an hour followed by washing three-times with binding buffer to remove the heparin. Cells were then incubated with ¹²⁵I-VEGF (10 ng/mL)±Heparin (1 μg/mL)±Avastin® nM) at pH 6.5 for 2 h at C. ¹²⁵I-VEGF bound to the ECM was determined by extracting the cells with high salt as described under Methods. Results displayed as averages of triplicate determinations±SD values.

FIGS. 2A and B show that Avastin® enhances VEGF binding to heparin, and inhibits binding to FN. FIG. 1 A shows streptavidin-coated 96-well microtiter plates were coated with biotin (1 μg/mL) or biotin-heparin (5 μg/mL) and incubated with ¹²⁵I-VEGF (10 ng/mL) alone or with Avastin® at the concentrations indicated (0-100 nM) for 2 h at 4° C. ¹²⁵I-VEGF bound to the plates was extracted and counted and the average of triplicate determinations±SD are presented. FIG. 1B shows testing of Avastin®'s ability to modulate VEGF binding to FN was by coating 96-well polystyrene plates with FN (10 μg/mL) in the presence of heparin (10 μg/mL) and incubating the FN with ¹²⁵I-VEGF (10 ng/mL) and the indicated range of Avastin® concentrations (0-100 nM) at pH 6.5 for 2 h at 4° C. The ¹²⁵I-VEGF bound to FN was extracted and quantified as described under Methods. Results displayed as averages of triplicates±SD.

FIGS. 3A-C show heparin binds VEGF-Avastin® complexes and enhances VEGF binding to Avastin®. FIG. 1A shows Avastin® incubated with biotin (1 μg/mL) or biotin-heparin (5 μg/mL) coated plates in the presence and absence of VEGF (10 ng/mL) for 2 h at 4° C., and then an ELISA protocol was used to detect bound Avastin®. The average values of triplicate determinations±SD are shown. Only conditions where VEGF and Avastin® were incubated with biotin-heparin coated plates produced a signal above background. FIG. 1B shows that to understand whether heparin influences VEGF binding to Avastin®, Avastin® (0.5 nM) or Fc-FGFR1 (0.5 nM) coated protein A plates were incubated with ¹²⁵I-VEGF (10 ng/mL) and the indicated concentration of heparin (0-100 μg/mL) for 2 h at 4° C. The ¹²⁵I-VEGF hound to the surface was extracted and measured. The average of triplicate values±SD are presented. FIG. 1C shows that to measure the Avastin® bound to FN, an ELISA protocol was followed after the FN coated surfaces were incubated with Avastin® (0-1.00 nM)±VEGF (10 ng/mL) for 2 h at 4° C. Bound Avastin® was quantified using relative absorbance values that are represented as the average±SD of triplicate determinations.

FIGS. 4A-B show Avastin®-mediated inhibition of VEGF binding to FN is reduced at acidic pH. FIG. 4A show ¹²⁵I-VEGF (10 ng/mL)±Avastin 12 were incubated with FN coated plates in binding buffer at the indicated pH (5-8) for 2 h at 4° C. After an incubation ¹²⁵I-VEGF hound to FN was extracted and counted. The average of triplicate values±SD are shown. FIG. 4B shows the values of ¹²⁵I-VEGF bound in the presence and absence of Avastin® were used calculate % VEGF binding inhibition using the equation:

$$\% VEGF binding inhibition = \frac{VEGF Bound ( Absence of Avastin ) - ( Presence of Avastin )}{VEGF Bound in the Absence of Avastin} * 100$$

FIG. 5 shows VEGF binding to Avastin® is reduced at acidic pH. ¹²⁵I-VEGF (10 ng/mL) binding to Avastin® (0.5 nM) coated onto protein A plates was measured at various pH's after a 2 h incubation at 4° C. ¹²⁵I-VEGF association to Fc-FGER1 (0.5 nM) coated plates was also measured to control for non-specific interactions. The average ¹²⁵I-VEGF bound (fmoles/well)±SD of triplicate determinations is shown.

FIG. 6 shows heparin enhances Avastin®-mediated inhibition of VEGF binding to FN at low pH. ¹²⁵I-VEGF (10 ng/mL)±Avastin® (1 nM)±heparin (1 μg/mL) were incubated with FN coated plates in binding buffer at pH 5.5 for 2 h at 4° C. The amount of ¹²⁵I-VEGF bound (fmoles/well) was measured in triplicate wells and is presented as the average±SD.

FIG. 7 shows heparin enhances VEGF binding to Avastin® at acidic pH. ¹²⁵I-VEGF (10 ng/mL) was incubated with an Avastin® (0.5 nM) coated Protein A plate at pH 5.5 and 7.5 in the presence and absence of heparin (10 μg/mL) for 2 h at 4° C. The amount of ¹²⁵I-VEGF bound (fmoles/well) was measured in triplicate wells and is presented as the average±SD.

FIGS. 8 A and B show heparin enhances Avastin® inhibition of VEGF binding to ECM and cell surface binding sites at low pH. PAEC FLK cells were pretreated with heparin (10 μg/mL) to expose VEGF binding sites for 1 h at 4° C. on ice, washed and then incubated with ¹²⁵I-VEGF (10 ng/mL)±heparin (1 μg/mL)±Avastin® (1 nM) in binding buffer at pH 5.5, 6.5, and 7.5 for 2 h at 4° C. on ice. The ¹²⁵I-VEGF bound to 8A ECM and 8B cell surface. were extracted and measured as described under Methods. The level of VEGF bound per well in each fraction are presented as the average of triplicate determinations±SD.

FIG. 9 shows that the combined addition of Avastin® with heparin inhibits VEGF-mediated activation of extracellular signal-regulated kinases (ERK) in endothelial cells. Serum-starved bovine aortic endothelial cells (3,300 cells/well) were treated with 25 ng/mL VEGF±1 nM Avastin®±10 μg/mL heparin for 10 and 30 min at pH 6.5. The cells were then fixed and probed with phospho-ERK 1° antibody (1:1000) and total ERK 1° antibody (1:1000) followed by Goat Anti-Rabbit 2° antibody (1:1000) and absorbance read on the spectrophotometer at 450 nm as well as 570 nm for background correction. VEGF-mediated activation of ERK. was calculated as phospho-ERK/total ERK in VEGF treated cells divided by that in the reciprocal non-VEGF treated cells. The data are the average of triplicate determinations±SD.

FIG. 10 is a schematic representation of heparin-Avastin® conjugation. Biotin-heparin and biotin-Avastin® will be bound to streptavidin (Scenario 1) or to streptavidin immobilized to agarose beads (Scenario 2). Alternatively, streptavidin will be directly conjugated to Avastin® and biotin-heparin bound to the complex (Scenario 3). All three scenarios rim to facilitate the formation of high affinity ternary complexes involving heparin, VEGF and Avastin®.

FIG. 11 shows that the co-localization of biotin-Avastin® and biotin-heparin onto a streptavidin leads to heparin-mediated increase in VEGF binding. Biotin-heparin was purchased from Millipore Sigma (Burlington, Mass.), and Avastin® was labeled with biotin using the EZ-Link™ sulfo-NHS-LC biotinylation kit from ThermoFisher Scientific (Waltham, MA) according to the manufacturer's directions. Biotin-Avastin® (1 nM) with a range of biotin-heparin concentrations was bound to streptavidin coated plates. ¹²⁵I-VEGF (25ng/mL) was incubated with the plates at pH 7.5. Bound ¹²⁵I-VEGF was extracted and counted and the average of triplicate determinations±SD are shown.

FIG. 12 shows that the ability of heparin to enhance VEGF binding to Avastin® is dependent on the density of streptavidin. A range of streptavidin concentrations were adsorbed onto a bacteriologic plate surface and then biotin-Avastin® (1 nM)±biotin-heparin (50 nM) were allowed to bind to the streptavidin surface. ¹²⁵I-VEGF (25ng/mL) was incubated with the plates and bound ¹²⁵I-VEGF was extracted and counted and the average of triplicate determinations±SD are shown.

FIG. 13 shows that biotin-heparin rescues VEGF binding to biotin-Avastin® on a streptavidin surface at acidic pH. Streptavidin (200 nM) was adsorbed onto plastic plates and biotin-Avastin® (1 nM), biotin-heparin (50 nM), or both were allowed to bind to the surface. ¹²⁵I-VEGF (10 ng/mL) in binding buffer at pH 5.5 or 7.5 was incubated with the surfaces and the VEGF hound to the plates was extracted and counted. The average of triplicate determinations±SD are shown.

FIGS. 14A and B show that co-localizing biotin-heparin with biotin-Avastin® through streptavidin is more effective at promoting VEGF binding to Avastin® than is soluble heparin. FIG. 14A shows buffer, biotin-Avastin® (1 nM), biotin-heparin (80 nM), or both biotin-Avastin® (1 nM) and biotin-heparin (80 nM) incubated with streptavidin coated plates overnight. ¹²⁵I-VEGF was incubated with the plate surfaces at pH 7.5 in the presence and absence of solubly heparin (80 nM) and the amount bound was extracted and counted. The average of triplicate determinations±SD are shown. FIG. 14B shows Biotin-Avastin® and a concentration range of biotin-heparin (0-100 nM) was bound to a streptavidin coated plate overnight. Then ¹²⁵I-VEGF binding to Avastin® was measured at pH 5.5 in the presence of a concentration range of soluble heparin (0-100 nM). The average of triplicate determinations±SD are shown.

FIGS. 15A and B show that co-localizing biotin-Avastin® and biotin-heparin on streptavidin inhibits VEGF-mediated endothelial cell migration. FIG. 15A shows streptavidin (200nM) was adsorbed onto polystyrene plate surfaces that were then incubated with buffer, biotin-Avastin® (1nM), biotin-heparin (50nM), or both biotin-Avastin® (1 nM) and biotin-heparin (50 nM). The coated plates were washed with buffer and then incubated ±VEGF (25ng/mL) for 2 h. Fluoroblock™ inserts containing serum-starved human umbilical vein endothelial cells (HUVECs) (50,000 cells/insert) were placed on top of each well and the cells allowed to migrate for 14-16 h. The cells were labeled with Calcein AM (4 μg/mL) and detected by fluorescence excitation at 485 am and emission at 530 nm with an auto cutoff of the bottom plate only at 515 nm (6 flashed/read). The relative fluorescence units of VEGF stimulated values divided by the reciprocal condition without VEGF are represented as “VEGF Stimulation (Fold)”. Shown are averages of triplicate determinations ±SEM. FIG. B shows a schematic representation of the cell migration experimental set up showing the various conditions tested and the result observed.

FIG. 16 shows that biotin-heparin binding to streptavidin conjugated to Avastin® leads to increased VEGF binding to Avastin®. Stretavidin was conjugated to Avastin® using the streptavidin conjugation kit from Abeam Inc (Cambridge, Mass.) following the manufacturer's instructions. Mixtures of buffer±streptavidin-Avastin® (5nM)±biotin-heparin (50 nM and 100 nM) were incubated overnight in solution and then incubated with a protein A plate to selectively bind the streptavidin-Avastin®. Then ¹²⁵I-VEGF (25 ng/mL) was incubated with the plate surfaces for 2 h at 4° C., and the VEGF bound to the plates was extracted and counted. The average of triplicate determinations±SD are presented.

The above-described and other features will be appreciated and understood by those skilled in the art from the following detailed description, drawings, and appended claims.

### DETAILED DESCRIPTION

The inventors have investigated the ability of Avastin® to influence VEGF binding to FN and to the ECM and VEGF receptors of endothelial cells, and have explored the ability of heparin to modulate this process. Using an array of cell-free and cell-based binding assays, the inventors found that Avastin® partially inhibited VEGF binding to purified FN and cell-based ECM, and that this activity was dramatically attenuated at acidic pH. Moreover, the pH-dependent loss of Avastin® inhibitory activity was correlated with a reduction of VEGF-Avastin® binding at acidic pH. Interestingly, the reduced Avastin®-VEGF binding at acidic pH was rescued by heparin as was the ability of Avastin® to inhibit VEGF binding to FN, ECM and cell surface receptors. The ability of heparin to enhance Avastin®-mediated inhibition of VEGF binding correlated with the ability of heparin to amplify Avastin®-inhibition of VEGF-induced activation of ERK in endothelial cells. Co-localizing Avastin® with heparin, using a biotin-streptavidin system, shows that the ability of heparin to enhance VEGF binding to Avastin® is amplified by bringing the two molecules (heparin and Avastin®) into close physical proximity of one another. Thus, the activities of heparin described herein are likely the reflection of the formation of a high affinity complex involving heparin, Avastin® and VEGF. Indeed, co-localizing biotin-heparin and biotin-Avastin® onto a streptavidin surface was significantly more effective at inhibiting VEGF-induced endothelial cell migration than either biotin-Avastin® or biotin-heparin alone.

These results suggest that the addition of heparin might expand the clinical utility of Avastin® and other anti-VEGF antibodies, anti-VEGF antibody fragments, or VEGF binding proteins that block its activity (anti-VEGF proteins). Taken together these findings highlight the importance of defining the range of interactions of an antibody target under a variety of conditions in order to fully predict antibody activity within a complex biological setting.

In an aspect, a method of treating a subject in need of treatment for pathological angiogenesis comprises administering to the subject a glycosaminoglycan linked either covalently or noncovalently to an anti-VEGF antibody, an anti-VEGF antibody fragment, an anti-VEGF protein, an anti- VEGF peptide, or an anti-VEGF aptamer.

In another aspect, a composition comprises a glycosaminoglycan linked either covalently or noncovalently to an anti-VEGF antibody, an anti-VEGF antibody fragment, an anti-VEGF protein, an anti-VEGF peptide, or an anti-VEGF aptamer.

In an aspect, the heparin and the anti-VEGF antibody, anti-VEGF antibody fragment, anti-VEGF protein, or anti-VEGF peptide are in a molar ratio of 1:0.001 to 1:10.

As used herein, glycosaminoglycan includes heparin, heparan sulfate, a non-heparin glycosaminoglycan such as dermatan sulfate, hyaluronic acid, chondroitin sulfate, and keratan sulfate, or an oligosaccharide synthesized chemically or derived from a glycosaminoglycan. The glycosoaminoglycans are optionally treated to add or remove sulfate groups, to add or remove acetylate groups, and or depolymerized to produce shorter oligosaccharides. The glycosaminoglycan can be modified chemically or enzymatically to generate oligosaccharides of specified length. The glycosaminoglycan can be modified chemically or enzymatically to remove anticoagulant activity.

Glycosaminoglycans (GAGs) belong to a highly heterogeneous class of macromolecules that contain repeating disaccharide units forming linear macromolecules. In general, each of the repeating units comprises a residue containing an aminosugar, that is glucosamine or galactosamine, and a uronic acid residue consisting of glucuronic acid or iduronic acid. The hydroxyl group at C (2), C (3), C (4) and C (6) and the amino group on C (2) may be substituted by sulfate groups. GAGs include the following compounds: heparin, heparan sulfate (HS), dermatan sulfate (DS), hyaluronic acid (HA), chondroitin sulfate (CS), and keratan sulfate.

Generally, in nature, a glycosaminoglycan is covalently attached to a protein core which often contains other glycosaminoglycans, e.g., a protein core may contain both heparan sulfate and chondroitin sulfate. Hyaluronic acid is not attached to a protein core.

Heparan sulfate (HS) is a linear polysaccharide that, in nature, is covalently attached to a protein core which often contains other glycosaminoglycans. When the protein core contains heparan sulfate, the entire molecule is referred to as a heparan sulfate proteoglycan (HSPG). Core proteins vary in size from 32 to 500 kDa.

Heparan sulfate macromolecules include 50-200 repeating disaccharide units (25-100 kDa). These disaccharide units consist of glucuronic acid (GlcA) or iduronic acid (IdoA) α-linked to N-acetylglucosamine (GlcNAc). Biosynthesis of HS occurs in the Golgi apparatus and is a complex process that begins with the stepwise addition of a xylose, two galactose molecules, and a GlcA to a serine residue on the core protein. Subsequently, GlcNAc is added committing the chain to HS synthesis. Following polymerization, a series of enzyme reactions results in regions of variable sulfation and acetylation. The exact pattern of these modifications can vary greatly between HS chains, and it is this variation that allows the many binding and regulatory properties of HS towards proteins.

Heparin is a molecule closely related to heparan sulfate as heparin also comprises polymers of repeating disaccharide units; D-glucosamine-L-iduronic acid and D-glucosamine-D-glucuronic acid. However, heparin contains relatively more iduronic acid than heparan sulfate and has a higher degree of sulfation.

Also contemplated are the use of derivatives of the above identified glycosaminoglycans. Derivatives include glycosaminoglycans that have been subjected to chemical and enzymatic modification, for example to remove or add sulfation and acetylation or to generate oligosaccharides of specified length.

Heterogeneity in heparin and other glycosaminoglycans results from variations in chain length, different carbohydrate backbone sequences, and the pattern and degree of sulfation. Studies have indicated that specific regions or “sequences” along heparin and other glycosaminoglycan chains allow for high affinity binding and modulation of a wide range of proteins.

The glycosaminoglycans such as heparin and heparan sulfate oligosaccharides can be obtained from natural sources. Alternatively, synthetic oligosaccharides or biomimetic chemicals can be used in place of naturally derived glycosaminoglycans, e.g., heparin. Means for isolation, identification, and quantitation of specific glycosaminoglycans are well known to those skilled in the art.

Glycosaminoglycans such as heparin, heparan sulfate and oligosaccharides of a specific sequence are used to inhibit VEGF in conjunction with anti-VEGF antibodies or other anti-VEGF proteins. Also contemplated is the use of derivatives of the above identified glycosaminoglycans in conjunction with anti-VEGF antibodies or other anti-VEGF proteins. Derivatives include glycosaminoglycans that have been subjected to chemical and enzymatic modification, for example to remove or add sulfation and acetylation or to generate oligosaccharides of specified length. These modifications can be achieved through chemical or enzymatic treatment of glycosaminoglycans by means know in the art.

In an aspect, the anti-VEGF antibody or fragment is bevacizumab (Avastiri®), ranibizumab (Lucentis®), brolucizumab, the anti-VEGF/anti-angiopoietin-2 bispecific antibody RG7716. Exemplary anti-VEGF proteins and peptides include aflibercept (Eylea®). Exemplary anti-VEGF aptamers include pegaptanib.

In an aspect, the glycosoaminolycan and the anti-VEGF antibody, anti-VEGF antibody fragment, anti-VEGF protein, or anti-VEGF peptide can be linked noncovalently using, for example, biotin-streptavidin. In this embodiment, the anti-VEGF antibody or anti-VEGF antibody and the glycosaminoglycan are both biotinylated and then bound to streptavidin, such as streptavidin agarose. Additional noncovalent binding strategies include avidin-biotin interaction, histidine-divalent metal ion interaction (e.g., Ni, Co, Cu, Fe), interactions between multimerization (e.g., dimerization) domains, glutathione S-transferase (GST)-glutathione interaction, and the like.

In an aspect, the glycosaminoglycan and the anti-VEGF antibody, anti-VEGF antibody fragment, anti-VEGF protein, or anti-VEGF peptide can be linked covalently using, for example, a “spacer”, “spacer group” or “spacer moiety” which refer to a substituent which is generally divalent and that couples the glycosaminoglycan and the anti-VEGF antibody, anti-VEGF antibody fragment, anti-VEGF protein, or anti-VEGF peptide.

A variety of conjugation methods and chemistries can be used to conjugate a glycosaminoglycan and the anti-VEGF antibody, anti-VEGF antibody fragment, anti-VEGF protein, or anti-VEGF peptide. Various zero-length, homo-bifunctional, and hetero-bifunctional crosslinking reagents can be used. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. Homo- and hetero-bifunctional reagents generally contain two identical or two non-identical sites, respectively, which may be reactive with amino, sulfhydryl, guanidino, indole, or nonspecific groups.

In some embodiments, the glycosoaminoglycan comprises an amino-reactive group for reacting with a primary amine group on the anti-VEGF antibody, anti-VEGF antibody fragment, anti-VEGF protein, or anti-VEGF peptide, or on a linker. Exemplary amino-reactive groups include, but are not limited to, N-hydroxysuccinimide (NHS) esters, imidoesters, isocyanates, acylhalides, arylazides, p-nitrophenyl esters, aldehydes, and sulfonyl chlorides.

In some embodiments, the glycosoaminoglycan comprises a sulfhydryl-reactive group, e.g., for reacting with a cysteine residue in the anti-VEGF antibody, anti-VEGF antibody fragment, anti-VEGF protein, or anti-VEGF peptide. Exemplary sulfhydryl-reactive groups include, but are not limited to, maleimides, alkyl halides, pyridyl disulfides, and thiophthalimides.

in other embodiments, carbodiimides soluble in both water and organic solvent, are used as carboxyl-reactive reagents. These compounds react with free carboxyl groups forming a pseudourea that can then couple to available amines, yielding an amide linkage.

In some aspects, a glycosoaminoglycan is conjugated to the anti-VEGF antibody, anti-VEGF antibody fragment, anti-VEGF protein, or anti-VEGF peptide using a homobifunctional crosslinker.

In an aspect, the homobifunctional crosslinker is reactive with primary amines. Homobifunctional crosslinkers that are reactive with primary amines include NHS esters, imidoesters, isothiocyanates, isocyanates, acylhalides, arylazides, p-nitrophenyl esters, aldehydes, and sulfonyl chlorides.

Non-limiting examples of homobifunctional NHS esters include disuccinimidyl glutarate (DSG), disuccinimidyl suberate (DSS), bis(sulfosuccinimidyl) suberate (BS), disuccinimidyl tartarate (DST), disulfosuccinimidyl tartarate (sulfo-DST), bis-2-(succinimidooxycarbonyloxy)ethylsulfone (BSOCOES), bis-2-(sulfosuccinimidooxycarbonyloxy)ethylsulfone (sulfo-BSOCOES), ethylene glycolbis(succinimidylsuccinate) (EGS), ethylene glycolbis(sulfosuccinimidylsuccinate) (sulfo-EGS), dithiobis(succinimidylpropionate (DSP), and dithiobis(sulfosuccinimidylpropionate(sulfo-DSP). Non-limiting examples of homobifunctional imidoesters include dimethyl malonimidate (DMM), dimethyl succinimidate (DMSC), dimethyl adipimidate (DMA), dimethyl pimelimidate (DMP), dimethyl suberimidate (DMS), dimethyl-3,3′-oxydipropionimidate (DODP), dimethyl-3,3′-(methylenedioxy)dipropionimidate (DMDP), dimethyl-,3′-(dimethylenedioxy)dipropionimidate (DDDP), dimethyl-3,3′-(tetramethylenedioxy)dipropionimidate (DTDP), and dimethyl-3,3′-dithiobispropionimidate (DTBP).

Non-limiting examples of homobifunctional isothiocyanates include p-phenylenediisothiocyanate (DITC), and 4,4′-diisothiocyano-2,2′-disulfonic acid stilbene (DIDS). Non-limiting examples of homobifunctional isocyanates include xylene-diisocyanate, toluene-2,4-diisocyanate, toluene-2-isocyanate-4-isothiocyanate, 3-methoxydiphenylmethane-4,4′-diisocyanate, 2,2′-dicarboxy-4,4′-azophenyldiisocyanate, and hexamethylenediisocyanate. Non-limiting examples of homobifunctional arylhalides include 1,5-difluoro-2,4-dinitrobenzene (DFDNB), and 4,4′-difluoro-3,3′-dinitrophenyl-sulfone. Non-limiting examples of homobifunctional aliphatic aldehyde reagents include glyoxal, malondialdehyde, and glutaraldehyde. Non-limiting examples of homobifunctional acylating reagents include nitrophenyl esters of dicarboxylic acids. Non-limiting examples of homobifunctional aromatic sulfonyl chlorides include phenol-2,4-disulfonyl chloride, and alpha-naphthol-2,4-disulfortyl chloride. Non-limiting examples of additional amino-reactive homobifunctional reagents include erythritolbiscarbonate, which reacts with amines to give biscarbamates.

In an aspect, the homobifunctional crosslinker is reactive with free sulfhydryl groups. Homobifunctional crosslinkers reactive with free sulfhydryl groups include, e.g., maleimides, pyridyl disulfides, and alkyl halides.

Non-limiting examples of homobifunctional maleimides include bismaleimidohexane (BMH), N,N′-(1,3-phenylene) bismaleimide, N,N′-(1,2-phenylene)bismaleimide, azophenyldimaleimide, and bis(N-maleimidomethyl)ether. Non-limiting examples of homobifunctional pyridyl disulfides include 1,4-di-3′-(2′-pyridyldithio) propionamidobutane (DPDPB). Non-limiting examples of homobifunctional alkyl halides include 2,2′-dicarboxy-4,4′-diiodoacetamidoazobenzene, alpha,alpha′-diiodo-p-xylenesulfonic acid, alpha,alpha′-dibromo-p-xylenesulfonic acid, N,N′-bis(b-bromoethyl)benzylamine, N,N′-di(bromoacetyl)phenylhydrazine, and 1,2-di(bromoacetyl)amino-3-phenylpropane.

In an aspect, a glycosoaminoglycan is conjugated to the anti-VEGF antibody, anti-VEGF antibody fragment, anti-VEGF protein, or anti-VEGF peptide using a heterobifunctional reagent. Exemplary heterobifunctional reagents include amino-reactive reagents comprising a pyridyl disulfide moiety; amino-reactive reagents comprising a maleimide moiety; amino-reactive reagents comprising an alkyl halide moiety; and amino-reactive reagents comprising an alkyl dihalide moiety.

Non-limiting examples of hetero-bifunctional reagents with a pyridyl disulfide moiety and an amino-reactive NHS ester include N-succinimidyl-3-(2-pyridyldithio)propionate (SPDP), succinimidyl 6-3-(2-pyridyldithio)propionamidohexanoate (LC-SPDP), sulfosuccinimidyl 6-3-(2-pyridyldithio)propionamidohexanoate (sulfo-LCSPDP), 4-succinimidyloxycarbonyl-alpha-methyl-alpha-(2-pyridyldithio)toluene (SMPT), divinyl sulfone (DVS), and sulfosuccinimidyl 6-alpha-methyl-.alpha.-(2-pyridyldithio)toluamidohexanoate(sulfa-LC-SMPT).

Non-limiting examples of heterobifunctional reagents comprising a maleimide moiety and an amino-reactive NHS ester include succinimidyl maleimidylacetate (AMAS), succinimidyl 3-maleimidylpropionate (BMPS), N-gamma-maleimidobutyryloxysuccinimide ester (GMBS)N-gamma-maleimidobutyryloxysulfosuccinimide ester (sulfo-GMBS) succinimidyl 6-maleimidylhexanoate (EMCS), succinimidyl 3-maleimidylbenzoate (SMB), m-maleimidobenzoyl-N-hydroxysuccinimide ester (MBS), m-maleimidobenzoyl-N-hydroxysulfosuccinimide ester (sulfo-MBS), succinimidyl 4-(N-maleimidomethyl)cyclohexane-1-carboxylate (SMCC), sulfosuccinimidyl 4-(N-maleimidomethyl)cyclohexane-1-carboxylate (sulfo-SMCC), succinimidyl 4-(p-maleimidophenyl)butyrate (SMPB), and sulfosuccinimidyl 4-(p-maleimidophenyl)butyrate (sulfo-SMPB).

Non-limiting examples of heterobifunctional reagents comprising an alkyl halide moiety and an amino-reactive NHS ester include N-succinimidyl-(4-iodoacetyl)aminobenzoate (STAB), sulfosuccinimidyl-(4-iodoacetyl)aminobenzoate (sulfo-SIAB), succinimidyl-6-(iodoacetyl)aminohexanoate (SIAX), succinimidyl-6-(6-((iodoacetyl)-amino)hexanoylamino)hexanoate (SIAXX), succinimidyl-6-(((4-(iodoacetyl)-amino)methyl)-cyclohexane-1-carbonyl)ami-nohexanoate (SIACX), and succinimidyl-4((iodoacetyl)-amino)methylcyclohexane-1-carboxylate (SLAC).

A non-limiting example of a hetero-bifunctional reagent comprising an amino-reactive NHS ester and an alkyl dihalide moiety is N-hydroxysuccinimidyl 2,3-dibromopropionate (SDBP). A non-limiting example of a hetero-bifunctional reagent comprising an alkyl halide moiety and an amino-reactive p-nitrophenyl ester moiety include p-nitrophenyl iodoacetate (NPIA).

Thermo Scientific, for example, has published a Crosslinking Technical Handbook that allows one to select a crosslinker with functional groups to bind the molecules of interest, select a spacer arm and select a desired solubility. The functional groups that are commonly targeted for bioconjugation include primary amines, sulfhydryls, carbonyls, carbohydrates and carboxylic acids.

Whatever means are used to conjugate the glycosoaminoglycan and the anti-VEGF antibody, anti-VEGF antibody fragment, anti-VEGF protein, or anti-VEGF peptide, such conjugation provides an improved therapy for the treatment of pathological angiogenesis. The ability of the anti-VEGF antibody, anti-VEGF antibody fragment, anti-VEGF protein, or anti-VEGF peptide to inhibit VEGF binding to FN and ECM is expected to be enhanced by the covalently linked glycosoaminoglycan.

In some embodiments, the disorder associated with pathological angiogenesis is an ocular disorder or a cell proliferative disorder. Exemplary ocular disorders include age-related macular degeneration (AMD), macular degeneration, macular edema, diabetic macular edema (DME) (including focal, non-center DME and diffuse, center-involved DME), retinopathy, diabetic retinopathy (DR) (including proliferative DR (PDR), non-proliferative DR (NPDR), and high-altitude DR), other ischemia-related retinopathies, retinopathy of prematurity (ROP), retinal vein occlusion (RVO) (including central (CRVO) and branched (BRVO) forms), CNV (including myopic CNV), corneal neovascularization, a disease associated with conical neovascularization, retinal neovascularization, a disease associated with retinal/choroidal neovascularization, pathologic myopia, von Hippel-Lindau disease, histoplasmosis of the eye, familial exudative vitreoretinopathy (FEVR), Coats disease, Norrie Disease, osteoporosis-Pseudoglioma Syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, conjunctivitis (including infectious conjunctivitis and non-infectious (e.g., allergic) conjunctivitis), Leber congenital amaurosis, uveitis (including infectious and non-infectious uveitis), choroiditis, ocular histoplasmosis, blepharitis, dry eye, traumatic eye injury, and Sjögren's disease. In specific aspects, the ocular disorder is AMD, DME, DR, or RVO. In an aspect, the ocular disorder is AMD, specifically wet AMD.

In the treatment of ocular disorders, the glycosamioglycan linked either covalently or noncovalently to the anti-VEGF antibody, anti-VEGF antibody fragment, anti-VEGF protein, or anti-VEGF peptide can be administered by intravitreal injection, transconjunctival intravitreal injection, subconjunctival injection, or as a topical ophthalmic composition.

“Topical ophthalmic compositions” includes but are not limited to solutions, suspensions, gels, ointments, sprays and the like. Ophthalmic compositions are advantageously in the form of ophthalmic solutions or suspensions (i.e., eye drops), ophthalmic ointments, or ophthalmic gels. Depending upon the particular form selected, the compositions may contain various additives such as buffering agents, isotonizing agents, solubilizers, preservatives, viscosity-increasing agents, chelating agents, antioxidizing agents, antibiotics, sugars, and pH regulators.

Examples of preservatives include, but are not limited to chlorobutanol, sodium dehydroacetate, benzalkonium chloride, pyridinium chlorides, phenethyl alcohols, parahydroxybenzoic acid esters, ben zethonium chloride, hydrophilic dihalogenated copolymers of ethylene oxide and dimethyl ethylene-imine, mixtures thereof, and the like. The viscosity-increasing agents may be selected, for example, from methylcellulose, hydroxyethylcellulose, carboxymethylcellulose, hydroxypropylmethylcellulose, polyvinyl alcohol, carboxymethylcellulose, chondroitin sulfate, and salts thereof. Exemplary solubilizers include, but are not limited to, polyoxyethylene hydrogenated castor oil, polyethylene glycol, polysorbate 80, and polyoxyethylene monostearate. Typical chelating agents include, but are not limited to, sodium edetate, citric acid, salts of diethylenetriamine pentaacetic acid, diethylenetriamine pentamethylenephosphonic acid, and stabilizing agents such as sodium edetate and sodium hydrogen sulfite.

Exemplary buffers include, but are not limited to borate buffers, phosphate buffers, carbonate buffers, acetate buffers and the like. The concentration of buffer in the ophthalmic compositions may vary from about 1 mM to about 150 mM or more, depending on the particular buffer chosen.

Exemplary cell proliferative disorders include cancers. Exemplary cancers include breast cancer, kidney cancer, cervical cancer, ovarian cancer, colorectal cancer, non-small cell lung cancer, non-Hodgkins lymphoma (NHL), prostate cancer, liver cancer, head and neck cancer, melanoma, mesothelioma, and multiple myeloma.

Specific cancers treatable with Avastin®, for example, include stage III or IV ovarian cancer (OC) after primary surgery; recurrent epithelial ovarian, fallopian tube, or primary peritoneal cancer (rOC); persistent, recurrent, or metastatic cervical cancer (CC); metastatic renal cell carcinoma (mRCC); recurrent glioblastoma (GBM); first-line non-squamous non-small cell lung cancer (NSCLC); or metastatic colorectal cancer (MCRC).

In the treatment of cancer, the glycosomainoglycan linked to an anti-VEGF antibody, anti-VEGF antibody fragment, anti-VEGF protein, or anti-VEGF peptide can be co-administered with an anti-cancer agent such as carboplatin, pegylated liposomal doxorubicin, topotecan, paclitaxel, gemcitabine, cisplatin, interferon alpha, 5-fluorouracil-based chemotherapy, fluoropyrimidine-irinotecan, fluoropyrimidine-oxaliplatin, or a combination comprising one or more of the foregoing.

In the treatment of ocular disorders, the glycosoaminoglycan linked either covalently or noncovalently to an anti-VEGF antibody, anti-VEGF antibody fragment, anti-VEGF protein, or anti-VEGF peptide can be administered parenterally in a sterile medium, either subcutaneously, or intravenously, or intramuscularly, or intrastemally, or by infusion techniques, in the form of sterile injectable aqueous or oleaginous suspensions. Depending on the vehicle and concentration used, the drug can either be suspended or dissolved in the vehicle. Advantageously, adjuvants such as a local anaesthetic, preservative and buffering agents can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

The invention is further illustrated by the following non-limiting examples.

### EXAMPLES

### Methods

Reagents: Streptavidin Coated 96-well plates and Protein A-coated plates were from Pierce (Rockford, Ill.). Avastin® (bevacizumab) was from Midwinter Solutions Ltd. (Staffordshire, UK) and Selleck Chemicals (Houston, Tex.). Human plasma Fibronectin (FN), heparin and biotin-heparin were from Millipore Sigma (Burlington, Mass.). Cell Culture agents such as Dulbecco's modified Eagle's medium (DMEM), Penicillin: Streptomycin (1:1) cocktail, and L-Glutamine were purchased from Corning (Tewkesbury, Mass.). Fetal Bovine Serum (FBS) was from Atlanta Biologics (Atlanta, Ga.). Recombinant human Vascular Endothelial Growth Factor 165 and Fc-FGFR1 chimeric protein were purchased from R&D Systems (Minneapolis, Minn.). ¹²⁵I-Bolton Hunter's Reagent was purchased from Perkin Elmer Life Science (Boston, Mass.) and was used to label the VEGF as described in the art. Blocker Casein in phosphate buffered saline (PBS) and BCA assay kit were obtained from ThermoFisher Scientific (Waltham, Mass.). Peroxidase-AffiniPure Donkey Anti-Human IgG, Fc (gamma) Fragment Specific was obtained from Jackson Immunoresearch (West Grove, Pa.). KPL TMB Microwell Peroxidase Substrate System was purchased from SeraCare (Milford, Mass.). EZ-Link™ sulfo-NHS-LC biotinylation kit were obtained from ThermoFisher Scientific (Waltham, Mass.). Streptavidin conjugation kit was obtained from Abeam Inc (Cambridge, Mass.). Anti-ERK1/2, anti-phospho ERK1/2 were purchased from Cell Signaling Technologies (Danvers, Mass.).

Heparin Binding Assays: Streptavidin coated 96-well plates were incubated with 5 μg/mL biotin-heparin or 1 μg/mL biotin in PBS overnight at 4° C. on a shaker (100 μL/well). Unbound biotin or biotin-heparin were washed away the next day, and combinations of ¹²⁵I-VEGF with and without Avastin® were added to each well in 100 μL of 0.1% BSA, 0.05% tween-20 in PBS (PBS-B-T). The treatments were incubated for 2 h at 4° C. on a shaker and then the unbound material was removed by washing each well three-times with binding buffer. Bound ¹²⁵I-VEGF was then extracted from the plate surface with 100 μL of 1N NaOH for 5 minutes followed by an additional 100 μL of 1N NaOH. The radioactivity in each sample was measured with a Cobra Auto-Gamma 5005γ-counter (Packard Instruments, Meridian, Conn.) for 3 minutes.

Avastin Binding Assays: Protein A coated plates were incubated with either Avastin® or Fc-FGFR-1 (0.5 nM; 100 μL/well) in PBS-B-T for 2 h at room temperature (RT) on a shaker. The unbound protein was removed by washing each well three, times with PBS-B-T and then ¹²⁵I-VEGF+/−Heparin was added to each well and incubated for 2 h on a shaker at RT. ¹²⁵I-VEGF bound to the surface of each well was then extracted two times with 100 μL of 1N NaOH for 5 minutes each. The radioactivity in each sample was read on a Cobra Auto-Gamma 5005γ-counter for 3 minutes.

Fibronectin Binding Assays: 96-well polystyrene plates were coated with a mixture of 10 μg/mL FN and 10 μg/mL heparin in PBS overnight at 4° C. on a shaker. The heparin was removed from the FN adsorbed to the plate surface by washing each well three times with PBS and the FN surfaces were incubated with combinations of ¹²⁵I-VEGF+/−Avastin® in 125 mM NaCl, 25 mM HEPES, 0.01% BSA (100 μL/well) for 2 h at RT. VEGF bound to the FN matrix was either extracted with 1N NaOH and counted (as described above) or an ELISA protocol was used to measure bound Avastin® levels. For ELISA, the wells were blocked with 1.5% BSA, 0.2% Casein in 1×PBS for 1 h at RT on the shaker. The wells were then incubated with 1:10,000 Donkey Anti-Human IgG in blocking buffer for 35 minutes. The wells were then incubated with TMB substrate for 10 minutes and stopped with IN phosphoric acid. The absorbance was read at 450 and 570 nm on a SpectraMax® plate reader. A570 nm values were subtracted from A450 nm values to account for background. The samples were collected in tubes and each well was then incubated two times with 100 μL of 1N NaOH for 5 minutes each and the bound ¹²⁵⁻I-VEGF was extracted. The radioactivity in each sample was read on a Cobra Auto-Gamma 5005γ-counter for 3 minutes.

VEGF Binding to Endothelial Cells: Porcine Aortic Endothelial cells (PAECs) (passage 4-15) that have been engineered to express VEGFR-2 (ELK cells) or transformed with an empty vector (EV ells) were a gift from Nader Rahimi at the Boston University School of Medicine. PAECs were maintained in DMEM containing 10% PBS, 1% penicillin/streptomycin cocktail, and 2 mM L-Glutamine. The cells were plated at 30,000 cells/well in 24-well plates and incubated overnight. The following day the media was changed to 0.1% FBS containing media and the cells allowed to incubate for an additional 24 h. After starvation, the cells were washed 3 times with cell binding buffer (DMEM, 25 mM HEPES, 0.1% BSA, pH 6.5) and incubated on ice for 10 minutes in the last wash to inhibit receptor internalization. Mixtures of ¹²⁵I-VEGF, heparin, and A vastin® in the cell binding buffer were added to the cells and incubated for 2 h at 4° C. The cells were washed three times with cell binding buffer to remove unbound ¹²⁵I-VEGF, and the ECM was extracted with a high salt buffer (1M NaCl, 25 mM HEPES) followed by PBS. The remaining cell bound ¹²⁵I-VEGF was extracted with 200 μL of 1N NaOH twice for 5 minutes each. The radioactivity in each sample was read on a Cobra Auto-Gamma 5005γ-counter for 3 minutes. This was accompanied by an MTT assay to quantify the cell density of both cell lines on a separate plate.

MTT Proliferation Assay: To evaluate the cell density 2 days after plating, cell number was determined using an MTT (3-(4, 5-dimethylthiazolyl-2)-2,5-diphenyltetrazolium bromide) Proliferation Assay kit (ATCC, Manassas, Va.). The protocol followed the manufacturer's instructions where the cells were washed and then incubated with the MTT reagent for 2 h at 37° C. on the shaker. This was followed by addition of detergent to each well. The cell extract was incubated for another 2 h at RT on the shaker. Absorbance was measured at 570 nm on the SpectraMax® plate reader. The ¹²⁵I-VEGF bound levels were normalized based on the cell MTT absorbance values.

Cell Signaling Assay: Phospho-ERK and total ERK were measured through enzyme linked immunosorbent assay. Bovind aortic endothelial cells were plated at 3,300 cells/ well into a 96 well plate with 200 μL of media per well and placed in the 37° C. +5% CO₂ incubator overnight. The following day, the wells were washed once with 1× PBS and replenished with 200 μL of 0.2% calf serum, 2mM L-Glutamine, 1% penicillin/streptomycin cocktail and incubated overnight. The following day, the cells were washed once with 1×DMEM, 25 mM HEPES, 1 mg/mL BSA, pH 6.5 then stimulated with 25 ng/mL VEGF±1 nM Avastin®±10 ug/mL Heparin for 10 and 30 minutes. The plates were then placed on ice and the wells were washed once with cold 1× PBS then incubated with 95% methanol on ice for 10 minutes. After the methanol incubation, the wells were washed with 1× PBS then incubated with 4% paraformaldehyde for 20 minutes on ice. The paraformaldehyde was discarded and the wells were washed 4× with 1× PBS and blocked with 3% BSA in 1× Tris-buffered saline (TBS) overnight at 4° C. on the shaker. Wells were washed once with 1× TBS then incubated with phospho-ERK 1° antibody (1:1000) as well as total ERK 1° antibody (1:1000) in 3% BSA-TBS for 1.5 h at RT on the shaker. The wells were then washed 3× with 1× TBS and then incubated with Goat Anti-Rabbit 2° (1:1000) in 3% BSA-TBS for 30 minutes at RT on the shaker. The wells were then washed 3× with 1× TBS, 0.1% Tween®-20 and then 3× with 1× TBS. The wells were then incubated with 100 uL of 1:1 TMB 2 Component Microweil Peroxidase Substrate System for 10 min at RT, and the reaction was stopped with IN phosphoric acid addition and the wells were read at 450 nm as well as 570 nm for background correction.

Conjugation of Avastin® with Biotin: Avastin® was labeled with biotin in accordance to the manufacturer's directions. The required volume for 200 μg of Avastin® (13.5 uL of 14.9 mg/mL) was diluted to a volume of 700 μL in 1× PBS and mixed with 9 mM sulfo-NHS-LC-biotin, in 50-fold molar excess. The mixtures were incubated for 1 h at RT. Meanwhile, retia spin desalting columns were equilibrated by adding 1 mL of PBS and centrifuging it through at 1000×g's for 2 minutes and this was repeated 3 more times. The incubated mixture was added to the column and allowed to absorb into the resin. The column was spun down at 1000×g's for 2 minutes and the flow through containing the conjugated reagent was collected and saved for future experiments.

Conjugation of Avastin® with Streptavidin: Avastin® was labeled with streptavidin in accordance to the manufacturer's instructions. A 100 μg quantity of Avastin® (1 mg/mL) was mixed with a 1:10 volume of modifier and mixed gently and the mixture was used to reconstitute the lyophilized streptavidin mix. The mixture was allowed to incubate at RT for 3 h and then the quencher reagent was added at a ratio of 1:10 to the antibody volume. The mixture was incubated for 30 minutes at RT and then stored at 4° C. for future experiments. The concentration of streptavidin conjugated A vastin was recalculated with the additional volumes and the original Avastin® quantity.

Endothelial Cell Migration Assay: Human umbilical vein endothelial cells (HUVECs) were purchased from Lonza (Walkersvile, Md.) and used at 70-80% confluence. Cells were washed twice with endothelial basal media (EBM) with 0.1% bovine serum albumin (BSA), then supplemented with 15 ml of EBM media and placed in the incubator for 5 h in 37° C. +5% CO₂. The cells were then trypsinized and resuspended in trypsin neutralizing solution once all the cells had lifted. The cells were spun down at 200 g's for 5 minutes and resuspended in EBM+0.1% BSA. The cells were counted, and 50,000 cells were placed on the top compartment of 8 μM Fluoroblok™ inserts (Corning, Corning, N.Y.) that had been coated with 0.1% porcine gelatin for 30 minutes at 37° C. +5% CO₂. Simultaneously, 24 well tissue culture plates were coated with 300 uL of 200 nM streptavidin in 1× PBS overnight at 4° C. The following day, the wells were washed once with 1× PBS and then incubated with 300 uL of 1× PBS+/−1 nM biotinylated Avastin®, +/−biotinylated heparin (concentration indicated in the Figures) overnight on the shaker at 4° C. The wells were incubated with 750 μLs of +/−25 ng/mL VEGF in pH specific buffer for 2 hours at 4° C. on the shaker. After VEGF was allowed to interact with the immobilized biotinylated Avastin® and heparin, the inserts containing the cells were transferred to the plates containing the chemoattractant. The cells were placed in the incubator at 37° C.+5% CO₂ for 14-16 h to allow the cells to migrate. After the migration period, the unattached cells at the top of the plate were removed, and then inserts were transferred to new wells containing 750 μL of 4 μg/mL, Calcein AM (Thermo Fisher Scientific, Waltham, Mass.). The plates were incubated for 1.5 hr in the incubator at 37° C.+5% CO₂ and, then read on a Spectramax® Fluorescence Plate Reader with excitation at 485 nm, emission at 530 nm and auto cutoff at 515 nm with bottom only plate read at 6 flashes/read.

### Example 1

### Avastin® Inhibits VEGF Binding to FN But Not to Heparin

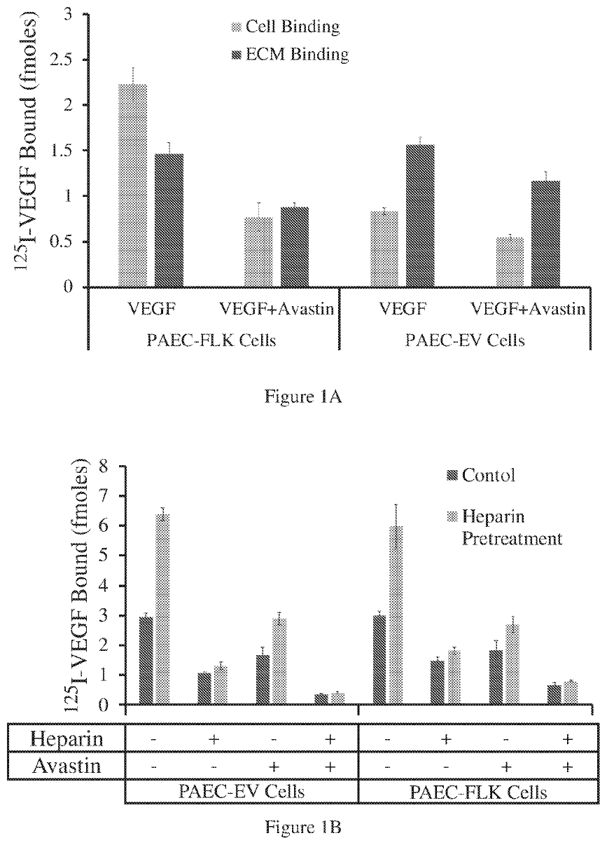

Avastin® is a monoclonal antibody against VEGF that is able to prevent VEGF binding to VEGFR-2. While this mechanism of action leads to inhibition of VEGFR-2 signaling, the complexity of VEGF interactions with other components on and around endothelial cells likely contributes to the inconsistent effectiveness of Avastin®. To examine the ability of Avastin® to modulate the array of VEGF binding interactions on endothelial cells, cells deficient in VEGF receptor expression (PAEC-EV) and those engineered to express VEGFR-2 (PAEC-ELK) were utilized. ¹²⁵I-VEGF binding to the extracellular matrix and the cell surface in the presence and absence of Avastin® were sequentially measured using selective extraction methods (FIG. 1). After ¹²⁵I-VEGF was allowed to bind to the endothelial cell monolayers, a high salt solution was used to extract the fraction of ¹²⁵I-VEGF bound to non-receptor components such as HS and FN (referred to as “ECM Binding”). The remaining bound ¹²⁵I-VEGF, which includes the fraction bound to VEGFR-2 on PAEC FLK cells, was extracted by solubilizing the cell layer in NaOH (“Cell Binding”). Avastin® reduced cell surface VEGF binding on PAEC FLK down to a level similar to that observed with non-receptor expressing PAEC-EV cells consistent with its known ability to block VEGF binding to VEGFR-2 (FIG. 1A). Interestingly, Avastin® also partially reduced binding in the ECM fraction in both PAEC-EV and PAEC-FLK cells, indicating that Avastin® binding to VEGF also interferes with non-receptor interactions (i.e., HS and FN).

To gain insight into the mechanism by which Avastin® affects VEGF binding to non-receptor binding sites, cells were pretreated with heparin to expose VEGF binding sites on FN. Thus, ECM binding of VEGF in cell layers subjected to heparin pretreatment will be enriched for VEGF bound to FN. When binding of ¹²⁵I-VEGF to the ECM was measured with PAEC-EV and PAEC-ELK cells, a significant increase (approximately 2-fold) in binding to cells that were pretreated with heparin compared to their respective non-pretreated controls was observed (FIG. 1B). Subsequently, including heparin in the binding buffer to compete for VEGF binding to FN caused a reduction in VEGF binding to the ECM that was more pronounced in the heparin pretreated cells. Interestingly, Avastin® also showed greater relative inhibition of VEGF binding to the ECM in cells that were pretreated with heparin suggesting that its ability to reduce ECM binding mainly lies in its ability to inhibit VEGF interactions with FN. The combination of Avastin® and heparin caused a further decrease in VEGF binding to the ECM. These results suggest that Avastin® may selectively interfere with VEGF binding to FN as opposed to HS sites on endothelial cells.

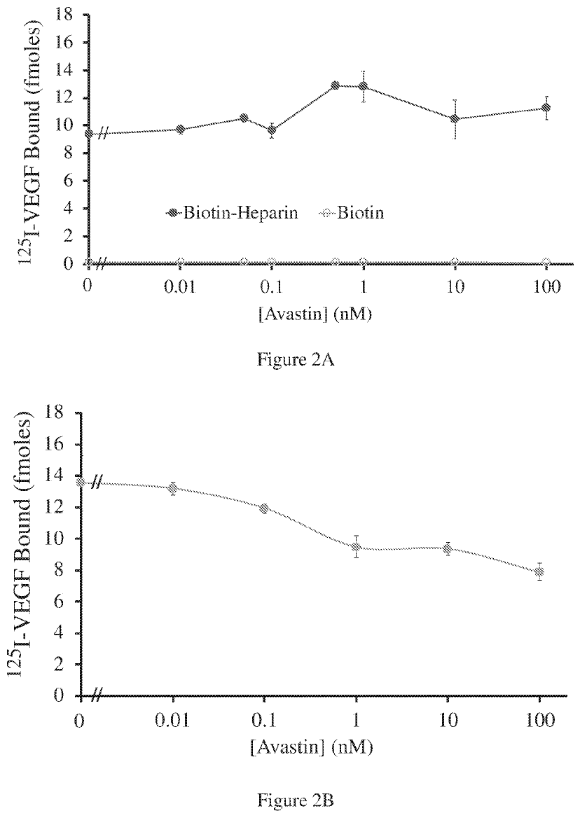

To distinguish the action of Avastin® on the two main classes of non-receptor binding sites for VEGF, cell-free assays using heparin (to represent heparan sulfate) and FN on microtiter plate surfaces were employed. ¹²⁵I-VEGF binding to streptavidin-biotin-heparin surfaces was measured and compared to streptavidin-biotin controls in the presence of a range of Avastin® concentrations (FIG. 2A). The presence of Avastin® caused a small (approximately 12-38%) increase in VEGF binding to heparin at concentrations above 0.5 nM, and none of the concentrations tested inhibited binding of VEGF. In contrast, Avastin® inhibited VEGF binding to FN adsorbed to polystyrene plates over the same concentration range.

### Example 2

### Avastin® Binds to Heparin and FN in the Presence of VEGF

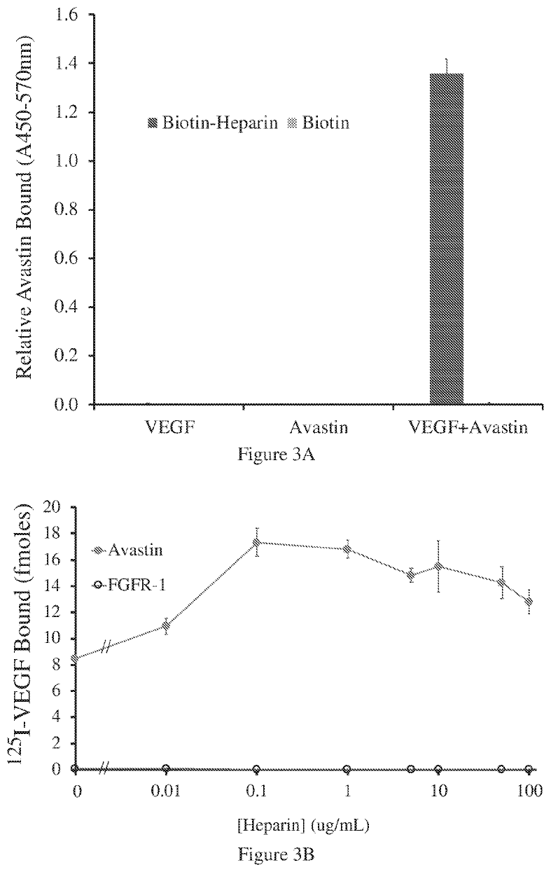

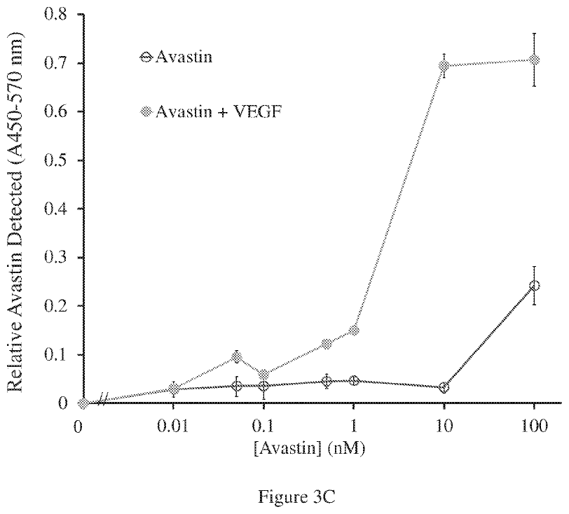

The fact that Avastin® was not able to inhibit VEGF binding to heparin suggests that the binding of VEGF to heparin and Avastin® are not mutually exclusive. Furthermore, the observation that VEGF binding to heparin was increased in the presence of Avastin® suggest that VEGF-Avastin® complexes might hind heparin with a higher affinity compared to VEGF alone. Alternatively, Avastin® could potentially increase VEGF binding to heparin-coated surfaces by simultaneously binding to heparin and VEGF. To gain insight into how Avastin® influences VEGF binding to heparin, Avastin® was incubated with heparin coated surfaces with and without. VEGF and the presence of Avastin® associated with the plate was measured with an HRP-linked secondary antibody using a standard ELISA protocol. VEGF alone was included as a negative control to determine the background level of secondary antibody binding to VEGF-heparin complexes. There was no detectable binding of Avastin® to the heparin plate in the absence of VEGF. However, the addition of VEGF resulted in a significant signal indicating that VEGF-Avastin® complexes are able to bind to heparin. The influence of heparin on VEGF binding to Avastin® was evaluated by immobilizing Avastin® to protein A coated plates and allowing soluble ¹²⁵I-VEGF to interact with the Avastin®-surface. A chimeric form of fibroblast growth factor, Fc-FGFR1, was also bound to the protein A plate as a control for potential non-specific interactions of VEGF with the Fe component of Avastin®. VEGF binding to immobilized Avastin® was significantly enhanced in the presence of heparin, with a maximal effect (>2-fold) being observed in the presence of 0.1 μg/mL, heparin (FIG. 3B). There was no detectable binding of ¹²⁵I-VEGF to the immobilized Fc-FGFR1 chimera. Thus, it appears that the binding of heparin to VEGF enhances VEGF binding to Avastin®, whereas VEGF binding to heparin is enhanced by Avastin®. Taken together these results indicate that heparin, VEGF, and Avastin® form a high affinity ternary complex, whereby the individual molecular interactions (VEGF with heparin, and VEGF with Avastin®) positively influence one another.

Avastin® binding to FN was also evaluated in the presence and absence of VEGF (FIG. 3C). While there was detectable Avastin® binding to the FN surface in the absence of VEGF, the levels were dramatically enhanced by VEGF, suggesting that Avastin® is able to bind to VEGF while it is complexed to FN.

### Example 3

### Inhibition of VEGF Binding to FN by Avastin® and Binding of VEGF to Avastin® are Decreased at Acidic pH

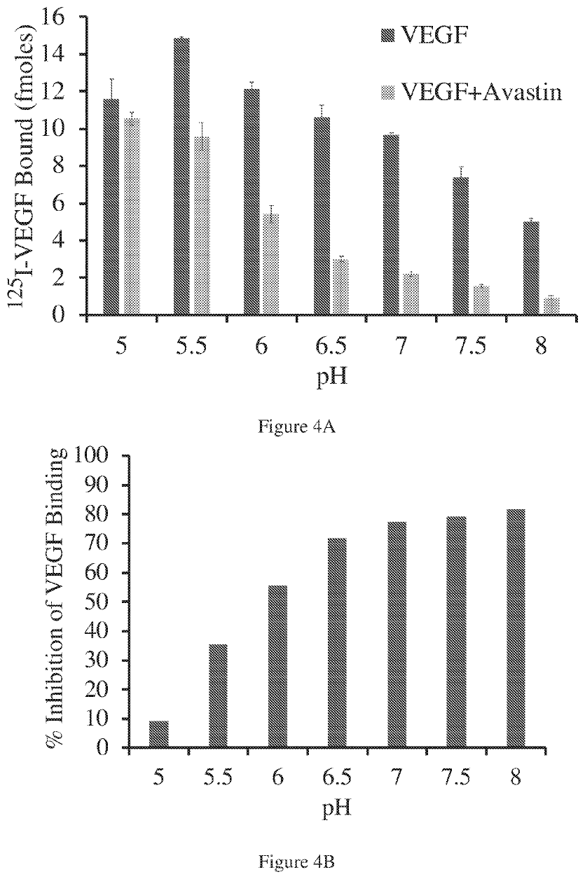

Previous studies have shown that VEGF binding to FN is enhanced at acidic pH values, thus, we investigated the ability of Avastin® to modulate VEGF binding to FN at a range of pH's (5 to 8). Consistent with previous studies, VEGF binding to FN increased as the pH decreased, but interestingly, the ability of Avastin® to inhibit VEGF-FN binding was reduced at acidic pH (FIG. 4). Whereas Avastin® inhibited VEGF binding by approximately 80% at pH 8, it only inhibited binding by approximately 10% at pH 5. The reduced ability of Avastin® to inhibit. VEGF binding to FN under acidic conditions could reflect an alteration in VEGF-Avastin® interactions under these low pH conditions. Thus, direct binding of VEGF to immobilized Avastin® was measured across this range of pH values and we observed an approximately 10-fold reduction in VEGF binding to Avastin® as the pH dropped from 8 to 5 (FIG. 5).

### Example 4

### Heparin Facilitates Avastin®-VEGF Binding and Enhances the Inhibition of VEGF Binding to FN

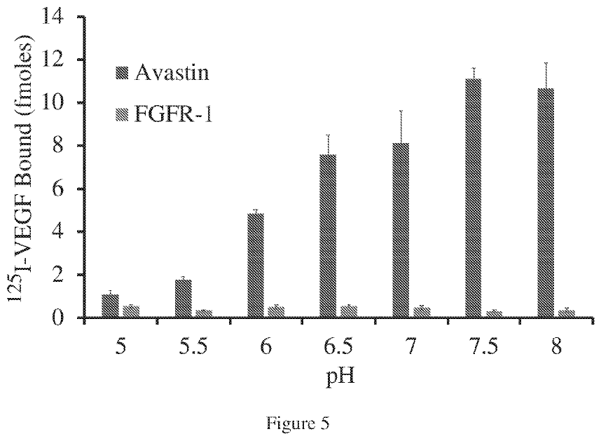

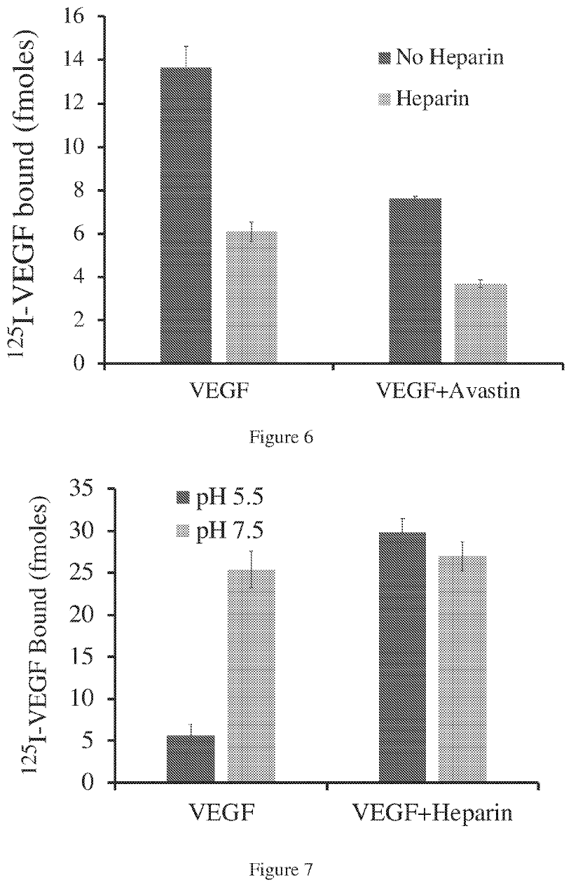

Since soluble heparin can inhibit VEGF binding to FN, the possibility that Avastin® and heparin might act together to produce greater inhibition was explored. Thus, VEGF binding to FN was measured in the presence of heparin, Avastin® and the combination of both inhibitors at pH 5.5 (FIG. 6). While each agent was able to partially inhibit VEGF binding under these conditions, the combination showed an additive response. Whereas Avastin® or heparin alone reduced binding by 44% and 55% respectively, the combination inhibited VEGF binding by 73%. These data indicate that heparin is able to facilitate Avastin®-mediated inhibition of VEGF binding under conditions (low pH) where Avastin® activity is compromised. To test whether heparin is able to enhance Avastin®-VEGF binding at low pH, binding of ¹²⁵⁻I-VEGF to immobilized Avastin® was measured in the presence and absence of heparin at pH 7.5 and 5.5 (FIG. 7). As observed above, the binding of ¹²⁵I-VEGF to Avastin® was significantly reduced at pH 5.5 compared to that at pH 7.5. However, there was no difference between VEGF-Avastin® binding under the two pH conditions when heparin was present suggesting that heparin is able to completely rescue VEGF-Avastin® binding under acidic conditions.

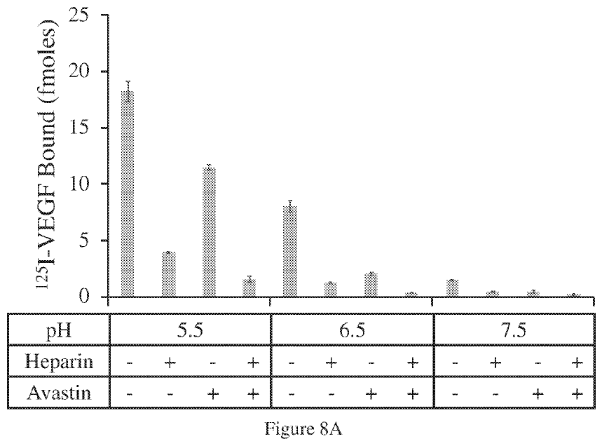

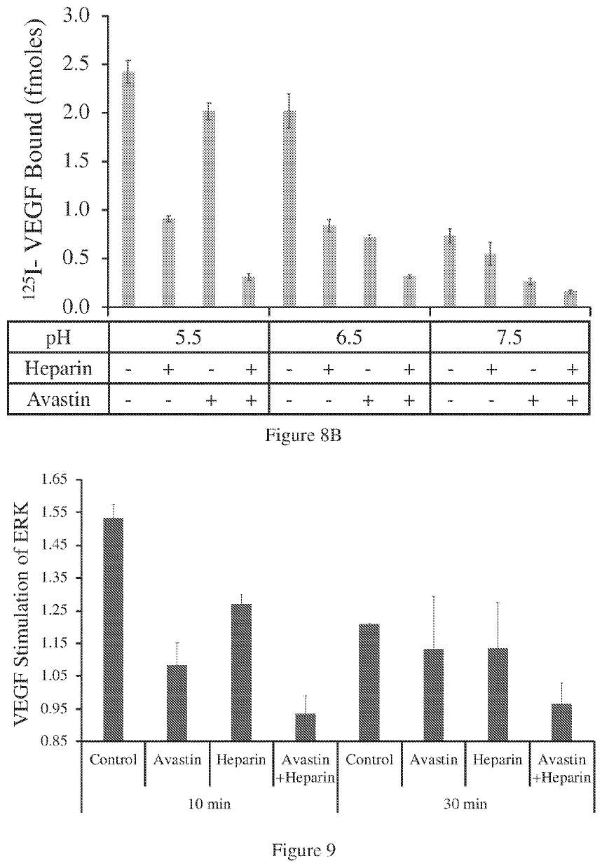

¹²⁵I-VEGF binding to PAEC FLK cells was measured to determine if the ability of heparin to overcome the effects of acidic conditions on Avastin®-mediated inhibition of VEGF binding to FN translates to enhanced inhibition of ECM and. VEGFR-2 binding within a complex cellular context. Measurements were conducted at pH 5.5, pH 6.5, and pH 7.5 as it was across this range of pH where we observed maximal alterations in Avastin® activity (FIGS. 4 and 5). As observed with purified FN, ECM binding of VEGF was enhanced at pH 5.5 compared to 6.5 and 7.5 (18.3 fmoles, at pH 5.5; 8.1 fmoles, at pH 6.5 and 1.4 fmoles, at pH 7.5 bound per well respectively), while the ability of Avastin® to inhibit VEGF binding was reduced (36.9% inhibition at pH 5.5; 74.3% inhibition at pH 6.5; 68.3% inhibition at pH 7.5) (FIG. 8A). However, the addition of heparin with Avastin® produced nearly identical levels of inhibition of VEGF binding to ECM under both pH 5.5 and pH 6.5 with a slightly lower level of inhibition at pH 7.5 (91.,5% inhibition at pH 5.5; 95.2% inhibition at pH 6.5; 85.7% inhibition at pH 7.5). These levels of inhibition were greater than those produced with either heparin or Avastin® alone. A similar pattern was observed when cell surface/VEGFR-2 binding was measured (FIG. 8B). Whereas Avastin® inhibited cell surface binding by 64.2% at pH 6.5 and 63.5 at pH 7.5, it only inhibited cell surface binding by 16.8% at pH 5.5. However, again, the addition of heparin with Avastin® dramatically enhanced inhibition under all pH conditions (87.1% inhibition at pH 5.5; 84.2% inhibition at pH 6.5; 78.2% inhibition at pH 7.5), greater than with heparin alone (62.5% at pH 5.5; 58.3% at pH 6.5; 25% at pH 7.5). While Avastin® was less active at low pH and showed greater inhibition at higher pH, heparin showed the opposite trend. Overall, the data indicate that the presence of heparin compensates for the loss of Avastin®-VEGF binding at low pH, thus, providing a means to restore Avastin®-mediated inhibition.

To determine if the ability of heparin to enhance Avastin®-mediated inhibition of VEGF binding translates into inhibition of VEGF activity on endothelial cells, we measured the activation of ERK, a key component of the VEGF-signaling pathway. Endothelial cells were treated with VEGF in the presence and absence of Avastin® and heparin (FIG. 9). VEGF-mediated activation (phosphorylation) of ERK in endothelial cells was partially reduced by Avastin or heparin alone, whereas the combination of Avastin and heparin resulted in a more dramatic inhibition at both time points tested. These results indicate that the ability of heparin to increase VEGF binding to Avastin® leads to enhanced inhibition of VEGF's pro-angiogenic activity.

### Discussion of Examples 1-4

Anti-VEGF therapies have improved outcomes for a variety of indications including cancer and retinal disease. One of the most widely used anti-VEGF drugs is bevacizumab (Avastin®), which was first given FDA approval in 2004 for combination use with chemotherapy. It has since been approved for a number of cancers and is also used off-label for the treatment of age-related macular degeneration and other retinal disorders. While Avastin® is a potent inhibitor of VEGF binding to VEGFR-2, it has been shown to bind to human retinal epithelial cells and human umbilical vein endothelial cells in the presence of VEGF. Without being held to theory, it is believed that A vastin®-VEGF complexes remain capable of binding to non-VEGFR-2 binding sites on cells and within the ECM. Indeed, these same studies also noted that VEGF is still able to bind neuropilin and heparin in the presence of Avastin®, and predicted that VEGF clearance was low due to being trapped by these VEGF binding molecules. Thus, in the present study, the inventors investigated the ability of Avastin® to modulate VEGF-binding to the ECM protein FN and we observed that Avastin® partially inhibited VEGF binding to FN (FIG. 2B), and that Avastin®-VEGF complexes were able to bind to FN (FIGS. 3C). Interestingly, the ability of Avastin® to inhibit VEGF binding to FN was enhanced by the addition of heparin, especially at acidic pH where Avastin®-VEGF interactions were dramatically attenuated (FIG. 7). It was further shown that the findings with purified components were consistent to those with endothelial cells expressing VEGFR-2. As observed with purified FN, Avastin® was significantly less able to reduce VEGF binding to the ECM of PAEC ELK cells at low pH and the addition of heparin was able to rescue Avastin® activity (FIG. 8). Taken together, the findings presented herein suggest that binding of VEGF to the ECM, specifically FN, may trap VEGF in locally acidic regions of tissue where it will interfere with the ability of Avastin® to effectively clear and inactivate VEGF.

When Avastin® binding to an FN matrix was measured in the presence and absence of VEGF, it was shown that Avastin® only associated with the FN surface when VEGF was present (FIG. 3C), suggesting that VEGF can hind to both Avastin® and FN allowing Avastin® to be retained within the ECM. It has been demonstrated that pretreatment of cells and FN with heparin leads to increased VEGF binding as a result of the ability of heparin to stably modify FN structure. However, the addition of soluble heparin along with VEGF reduced VEGF binding to the ECM, in the presence or absence of heparin pretreatment (FIG. 1B) and showed an additive effect with Avastin® (FIG. 1). Interestingly, heparin enhanced VEGF binding to Avastin®, and Avastin® enhanced VEGF binding to heparin, suggesting that Avastin®, VEGF and heparin form a cooperative high affinity ternary complex. These findings suggest the potential that a composite of heparin and Avastin® might function as a potent VEGF inhibitory agent that can both target VEGFR-2 and ECM binding.

While it is known that VEGF binding to the FN matrix is higher at lower pH conditions, these same conditions are also connected to triggering proangiogenic signals. The growth of tumors beyond a 1 mm diameter leaves the tumor center deprived of nutrients which not only leads to low O₂ levels, but also to low extracellular pH conditions. As these low pH conditions progress, the probability of VEGF binding to FN increases. This process can lead to enhanced VEGF deposition within the tumor microenvironment leading to a highly directive biochemical gradient. FN matrix bound VEGF may then become available to bind and activate VEGFR-2 on endothelial cell surfaces. Indeed, it has been demonstrated that VEGFR-2 and FN can simultaneously bind to VEGF suggesting a mechanism by which cells can respond to ECM associated VEGF. While anti-VEGF therapies such as Avastin® are able to target VEGF to prevent its binding to VEGFR-2, the inventors have shown that VEGF binding to FN is not completely inhibited in the presence of Avastin® (FIG. 2B), and that Avastin® can associate with FN in the presence of VEGF. Thus, unlike with VEGFR-2, Avastin® binding to VEGF does not appear to block the FN binding site on VEGF, but instead appears to reduce the affinity of the VEGF-FN interaction. Additionally, low pH conditions hinder VEGF and Avastin® binding (FIG. 5) which limits the ability of Avastin® to inhibit VEGF binding to ECM and VEGFR-2 (FIG. 8B). These findings identify an intrinsic limitation in the Avastin® mechanism of action that may hinder activity under certain conditions.

The prospects of treating disease by targeting pathological angiogenesis has become a reality that has extended and improved the lives of patients. In particular, the use of anti-VEGF antibodies and other binding proteins targeting VEGF have clearly demonstrated the value of targeting VEGF for antiangiogenic therapy. Unfortunately, most antiangiogenic drugs, such as Avastin®, have shown inconsistent results and somewhat limited clinical success in certain settings. ®The findings herein showing that Avastin® was not able to block VEGF binding to the ECM, specifically to FN, suggest that VEGF-ECM interactions may contribute to limiting Avastin® activity within a complex tissue environment. Moreover, the discovery that Avastin® binding to VEGF was dramatically reduced at acidic pH while binding to the ECM was enhanced suggests that this limitation might be compounded in hypoxic and locally acidic tissue regions. The observation that heparin rescued the pH-dependent loss of Avastin®-VEGF binding and was able to enhance the inhibitory effects of Avastin® on VEGF binding to FN and VEGFR-2 suggests the intriguing possibility that heparin, or other potentially more specific polysaccharides, might be used in combination with Avastin® to improve its clinical utility.

### Example 5

### Generation and Evaluation of Heparin-Avastin® Conjugates

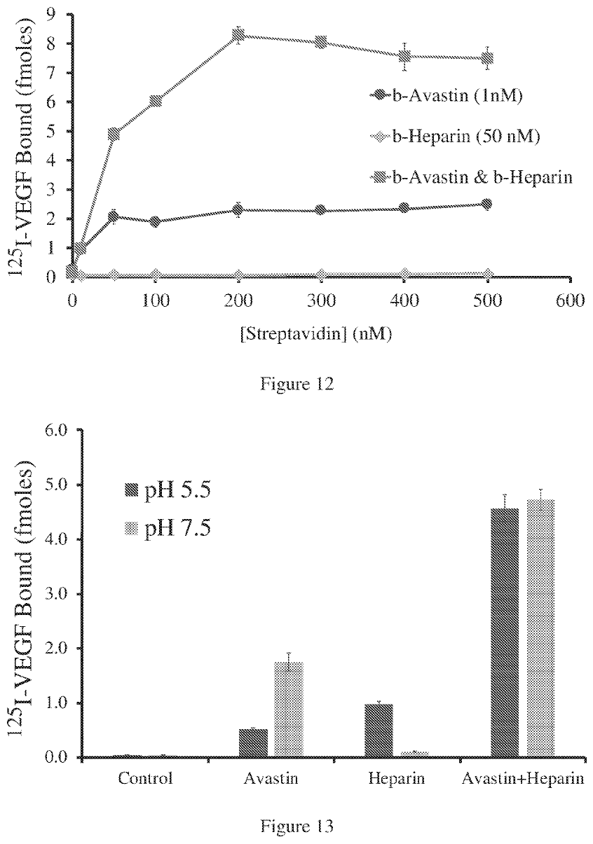

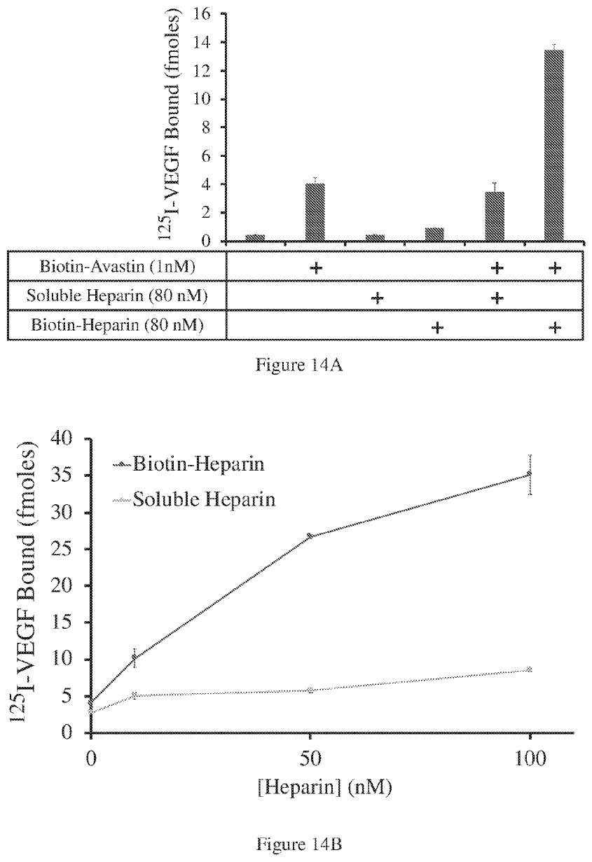

To facilitate the synergistic binding of heparin and Avastin® to VEGF as a means of blocking VEGF activity, the two compounds were linked through the use of the high affinity interaction between biotin and streptavidin. Biotin was covalently linked to Avastin® using standard amine chemistry following the manufacturer's instructions for the EZ-Link™ Biotinylation Kit (ThermoFisher). Heparin, end labeled with biotin, was purchased from Sigma. “Proof of concept” experiments were conducted with the two compounds by utilizing streptavidin-coated plastic microtiter plates. Biotin-Avastin® and biotin-heparin were mixed in solution at ratios from 1:0 to 1:1.00 and allowed to hind to streptavidin coated microplates. Streptavidin is a tetrameric protein that binds biotin with such a high affinity that the interaction is considered “permanent”. The streptavidin binding site surface density is estimated to be approximately 100 pmol/cm² and, assuming that biotin-Avastin® (density of approximately 0.3 pmol/cm²) bound randomly to the streptavidin surface, we predict that relatively high biotin-heparin concentrations would result in the two molecules coming into close proximity to one another. Indeed, as seen in FIG. 11, VEGF binding to Avastin® was dramatically increased (up to nearly 8-times that without heparin) at biotin-heparin concentrations between 10-100 nM (surface densities of 3-30 pmol/cm²). To further support the conclusion that the ability of biotin-heparin to enhance VEGF binding to surface immobilized biotin-Avastin® is the result of the two molecules being brought in close proximity, the density of streptavidin on the plate surface was varied (FIG. 12). As the concentration of streptavidin reached 50 nM, maximal binding of VEGF to biotin-Avastin® alone was observed, indicating that there were sufficient sites available for all the biotin-Ayastin® to bind to the streptavidin surface. However, the ability of biotin-heparin to increase VEGF binding to biotin-Avastin® was not observed until streptavidin was present at 200 nM and above, indicating the need for a higher density of streptavidin sites on the plate surface to increase the probability that biotin-heparin would be hound close enough to biotin-Avastin® to facilitate VEGF binding. The biotin-streptavidin system was also used to demonstrate that the co-localization of heparin and. Avastin® also replicates the ability of heparin to rescue the pH dependent binding of VEGF to Avastin® (FIG. 13). Furthermore, the ability of heparin to enhance VEGF binding to Avastin® at neutral and acidic pH was amplified by co-localization of biotin-heparin with biotin-Avastin® on the streptavidin surface compared to that observed with similar concentrations of freely soluble heparin (FIG. 14). Taken together, these findings demonstrate that the co-localization of heparin and Avastin® is a means to create a high affinity VEGF binding complex.

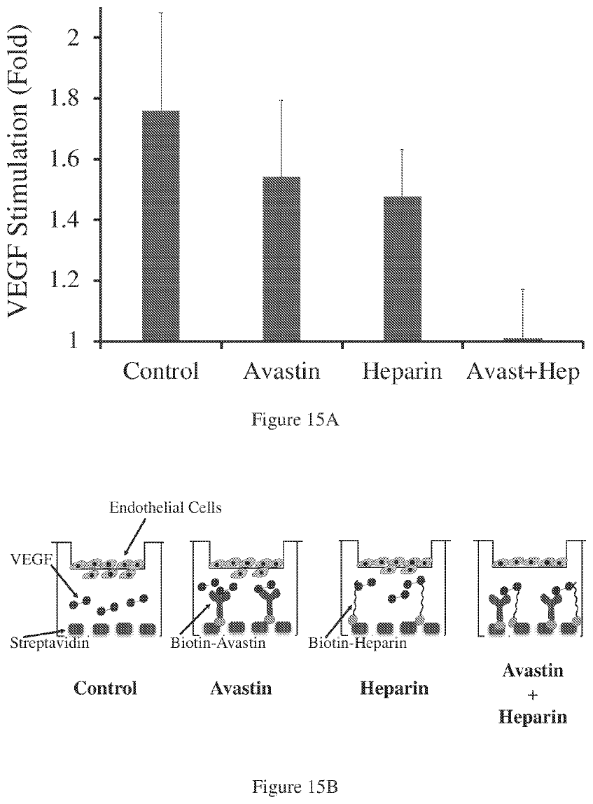

VEGF-stimulation of human endothelial cell migration was measured to determine if the enhanced binding of VEGF by biotin-Avastin® when complexed with biotin-heparin via streptavidin translates to increased inhibition of the biological activity of VEGF (FIG. 15). Streptavidin coated surfaces were complexed with biotin-Avastin®, biotin-heparin or both molecules and the ability of VEGF to stimulate the migration of HUVECs through a porous membrane was measured. The presence of biotin-Avastin® or biotin-heparin alone resulted in an approximately 30% and 35% reduction in VEGF stimulation of migration respectively. The combined addition of biotin-Avastin® with biotin-heparin resulted in complete inhibition of VEGF stimulation. These data demonstrate that the co-localization of Avastin® and heparin via streptavidin enhances the VEGF inhibitory activity of either molecule alone.

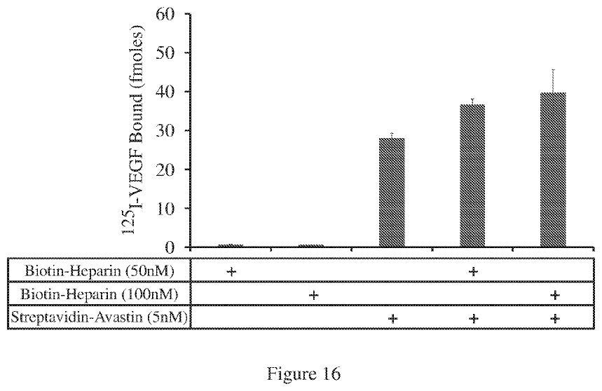

To further evaluate approaches to create soluble heparin-Avastin® conjugates, Avastin was covalently linked to streptavidin and then allowed to interact with biotin-heparin in solution. Complexes containing Avastin® were selectively isolated through binding to protein A via the common domain on Avastin®. ¹²⁵I-VEGF binding to the isolated streptavidin-Avastin® showed a biotin-heparin-mediated increase, indicating that biotin-heparin had become associated with the streptavidin-Avastin® and was able to enhance VEGF-Avastin® binding in this formation (FIG. 16).

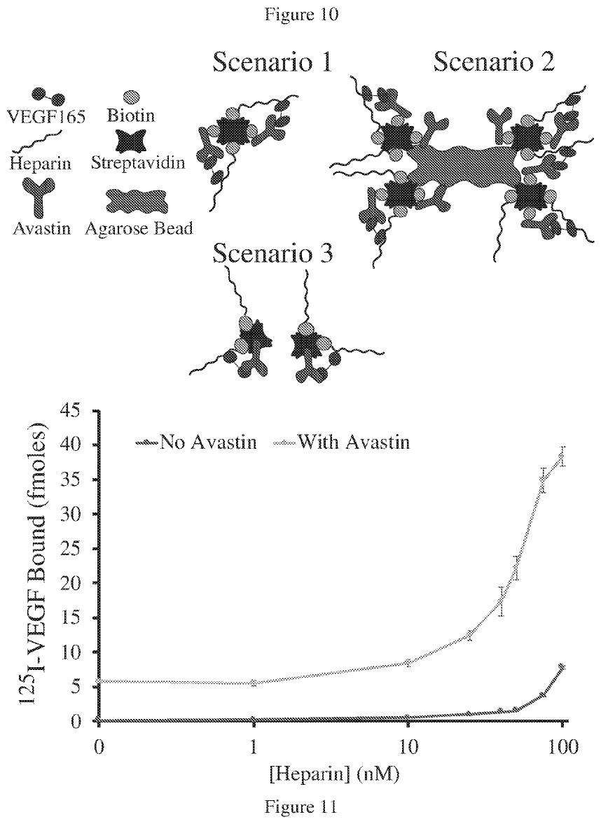

To evaluate the function of the heparin-Avastin® conjugates as VEGF inhibitors, we will prepare soluble conjugates by binding biotin-heparin and biotin-Avastin® to soluble streptavidin (FIG. 10, Scenario 1). Complexes that contain both heparin and Avastin® will be purified using a two-step affinity chromatography process involving anion exchange (Q-Sepharose®) to select for heparin followed by protein A-Sepharose® to select for Avastin®. The heparin-Avastin® conjugates will then be tested for their ability to inhibit VEGF binding to endothelial cells expressing VEGF receptor 2, and to the extracellular matrix of these cells in comparison to the activity of either compound linked to streptavidin alone. Similar VEGF binding assays in the presence and absence of the conjugates will be conducted with purified fibronectin. We expect to observe that the conjugation of heparin and Avastin® via streptavidin will facilitate the formation of the high affinity VEGF-heparin-Avastin® ternary complex that will lead to a dramatic increase in the ability to inhibit VEGF binding across a wide range of pH. After evaluating the ability of the heparin-Avastin® conjugate to inhibit VEGF binding, its ability to inhibit VEGF receptor signaling and cellular response (proliferation, migration and tube formation) compared to equal molar concentrations of Avastin® alone will also be evaluated.

The activity of biotin-heparin and biotin-Avastin® co-immobilized onto agarose beads (FIG. 10, Scenario 2) will also be explored. By immobilizing heparin and Avastin® onto agarose beads, high density VEGF binding sites will be created which will allow for trapping of VEGF. Thus, VEGF bound to heparin-Avastin® complexes that dissociate from these binding sites will encounter and bind nearby heparin and Avastin® leading to prolonged capture. Biotin-heparin and biotin-Avastin® will be hound to streptavidin-agarose beads and the beads added in suspension into culture media to measure their ability to inhibit VEGF binding and activity in endothelial cells. The activity of the heparin-Avastin® beads will be compared to the activity of equal quantities of heparin and Avastin® added in solution. We expect to find that the immobilized format will lead to much greater VEGF inhibition.

While we will initially evaluate heparin-Avastin® conjugates prepared with a 1:1 molar ration of each component, inhibition of VEGF may be greater under different conditions. Hence, we will also conduct the above studies using a range of heparin:Avastin® ratios. We anticipate that the ideal heparin:Avastin® ratio will be between 1:0.001 and 1:10.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of treating a subject in need of treatment for pathological angiogenesis, comprising administering to the subject a glycosaminoglycan linked either covalently or noncovalently to an anti-VEGF antibody, an anti-VEGF antibody fragment, an anti-VEGF protein, and anti-VEGF peptide, or an anti-VEGF aptamer.

2. The method of claim 1, wherein the glycosaminoglycan and the anti-VEGF antibody, an anti-VEGF antibody fragment, anti-VEGF protein, anti-VEGF peptide or anti-VEGF aptamer are in a molar ratio of 1:0.001 to 1:10.

3. The method of claim 1, wherein the glycosaminoglycan is heparin, heparan sulfate, a non-heparin glycosaminoglycan, or an oligosaccharide synthesized chemically or derived from a glycosaminoglycan.

4. (canceled)

5. The method of claim 1, wherein the glycosaminoglycan has been modified chemically or enzymatically to remove or add sulfation, to remove or add acetylation, or to remove or add anticoagulant activity.

6. (canceled)

7. (canceled)

8. The method of claim 1, wherein the anti-VEGF antibody, an anti-VEGF antibody fragment, anti-VEGF protein, anti-VEGF peptide, or anti-VEGF aptamer is bevacizumab, ranibizumab, brolucizumab, the anti-VEGF/anti-angiopoietin-2 bispecific antibody, RG7716, aflibercept, or pegaptanib.

9. The method of claim 1, wherein the pathological angiogenesis is associated with an ocular disorder or a cell proliferative disorder.

10. (canceled)

11. The method of claim 9, wherein the ocular disorder is age-related macular degeneration (AMD), macular degeneration, macular edema, diabetic macular edema (DME), retinopathy, diabetic retinopathy (DR), ischemia-related retinopathies, retinopathy of prematurity (ROP), retinal vein occlusion, CNV, corneal neovascularization, a disease associated with retinal/choroidal neovascularization, pathologic myopia, von Hippel-Lindau disease, histoplasmosis of the eye, familial exudative vitreoretinopathy (FEVR), Coats' disease, Norrie Disease, Osteoporosis-Pseudoglioma Syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, conjunctivitis, Leber congential amaurosis, uveitis, choroiditis, ocular histoplasmosis, blepharitis, dry eye, traumatic eye injury, or Sjögren's disease.

12. The method of claim 11, wherein the glyosaminoglycan linked either covalently or noncovalently to the anti-VEGF antibody, anti-VEGF antibody fragment, anti-VEGF protein, anti-VEGF peptide, or anti-VEGF aptamer is administered by intravitreal injection, transconjunctival intravitreal injection, subconjunctival injection, or a topical ophthalmic composition.

13. The method of claim 9, wherein cell proliferative disorder is a cancer and wherein the cancer is breast cancer, kidney cancer, cervical cancer, ovarian cancer, colorectal cancer non-small cell lung cancer, non-Hodgkins lymphoma (NHL), prostate cancer, liver cancer, head and neck cancer, melanoma, mesothelioma, or multiple myeloma.

14. (canceled)

15. The method of claim 13, wherein the cancer is stage III or IV ovarian cancer (OC) after primary surgery; recurrent epithelial ovarian, fallopian tube, or primary peritoneal cancer (rOC); persistent, recurrent, or metastatic cervical cancer (CC); metastatic renal cell carcinoma (mRCC); recurrent glioblastoma (GBM; first-line non-squamous non-small cell lung cancer (NSCLC); or metastatic colorectal cancer (MCRC).

16. The method of claim 13, further comprising administering carboplatin, pegylated liposomal doxorubicin, topotecan, paclitaxel, gemcitabine, cisplatin, interferon alpha, 5-fluororacil-based chemotherapy, fluoropyrimidine-irinotecan, fluoropyrimidine-oxaliplatin, or a combination comprising one or more of the foregoing.

17. The method of claim 13, wherein the glycosaminoglycan linked either covalently or noncovalently to the anti-VEGF antibody, anti-VEGF antibody fragment, anti-VEGF protein, or anti-VEGF peptide is administered parenterally.

18. The method of claim 1, wherein the glycosaminoglycan linked either covalently or noncovalently anti-VEGF antibody, anti-VEGF antibody fragment, anti-VEGF protein, or anti-VEGF peptide, or anti-VEGF aptamer through a streptavidin-biotin interaction, an avidin-biotin interaction, a histidine-divalent metal ion interaction, and interactions between multimerization domains, or a glutathione S-transferase (GST)-glutathione interaction.

19. The method of claim 1, wherein the glycosaminoglycan is covalently linked to the anti-VEGF antibody, anti-VEGF antibody fragment, anti-VEGF protein, or anti-VEGF peptide, or anti-VEGF aptamer through primary amines sulfhydryl, carbonyl, carbohydrate, and/or carboxylic acid functional groups, or through a homo-bifunctional or hetero-bifunctional reagents reactive with amino, sulfhydryl, guanidino, indole, nonspecific groups, or a combination thereof.

20. (canceled)

21. A composition comprising a glycosaminoglycan linked either covalently or noncovalently to an anti-VEGF antibody, anti-VEGF antibody fragment, anti-VEGF protein, or anti-VEGF peptide, or anti-VEGF aptamer.

22. The composition of claim 21, wherein the glycosaminoglycan and the anti-VEGF antibody, anti-VEGF antibody fragment, anti-VEGF protein, or anti-VEGF peptide, or anti-VEGF aptamer are in a molar ratio of 1:0.001 to 1:10.

23. The composition of claim 21, wherein the glycosaminoglycan is heparin, heparan sulfate, a non-heparin glycosaminoglycan, or an oligosaccharide synthesized chemically or derived from a glycosaminoglycan.

24. (canceled)

25. The composition of claim 21, wherein the glycosaminoglycan has been modified chemically or enzymatically to remove or add sulfation, to remove or add acetylation, or to remove or add anticoagulant activity.

26. (canceled)

27. (canceled)

28. The composition of claim 21, wherein the anti-VEGF antibody, anti-VEGF antibody fragment, anti-VEGF protein, or anti-VEGF peptide, or anti-VEGF aptamer is bevacizumab, ranibizumab, brolucizumab, the anti-VEGF/anti-angiopoietin-2 bispecific antibody, RG7716, aflibercept, or pegantanib.
